(19) United States
(12) Patent Application Publication
(10) Pub. No.: US 2009/0264753 A1
(43) Pub. Date: Oct. 22, 2009

(54) METHOD & SYSTEM FOR MULTI-MODALITY IMAGING OF SEQUENTIALLY OBTAINED PSEUDO-STEADY STATE DATA

(75) Inventors: Gustav K. von Schulthess, Zurich (CH); Eugene Saragnese, Delafield, WI (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, Schenectady, NY (US)

(21) Appl. No.: 12/107,648

(22) Filed: Apr. 22, 2008

(51) Int. Cl. A61B 5/00

(52) U.S. Cl. 600/431

(57) ABSTRACT

Methods, protocols and systems are provided for multi-modality imaging based on pharmacokinetics of an imaging agent. An imaging agent is introduced into a subject, and is permitted to collect generally in a region of interest (ROI) in the subject until attaining a pseudo-steady state (PSS) distribution within the ROI. The imaging agent records a first functional state of the ROI at a given point in time. A first image data set is obtained with a first imaging modality during a first acquisition time interval that occurs prior or proximate in time with the PSS time interval. The subject is transferred from the first imaging modality to a second imaging modality during a transfer time interval that overlaps the PSS time interval. Once transfer is complete, a second image data set is obtained with the second imaging modality during a second acquisition time interval that also overlaps the PSS time interval in which the imaging agent maintains the PSS distribution in the ROI. In accordance with a protocol, the transfer time interval and second acquisition time interval substantially fall within the PSS time interval. The imaging agent collects in the ROI during an uptake time interval which may or may not precede the time interval during which first imaging modality obtains at least a portion of the first image data set. The second image data set is obtained while the imaging agent persists in the ROI at the PSS distribution reflective of the first functional state even after the ROI is no longer in the first functional state.

### BACKGROUND OF THE INVENTION

Embodiments of the present invention generally relate to systems, protocols and methods for multi-modality imaging that utilize imaging contrast agents or radiopharmaceutical agents to obtain image data sets.

Today, a wide variety of imaging modalities exist for scanning various properties and characteristics of subjects. Examples of such imaging systems include positron emission tomography (PET), single photon emission computed tomography (SPECT), magnetic resonance imaging (MRI), computed tomography (CT), static X-ray imaging, dynamic X-ray imaging (fluoroscopy), ultrasound imaging, and optical imaging. Traditionally, the foregoing imaging systems were constructed and operated entirely separate and independent of one another. When operated independently, one imaging system would obtain one type of image data set representative of the subject at one point in time, while another imaging system would obtain another type of image data set representative of the subject at another point in time.

More recently, imaging systems have been proposed and developed that partially or fully integrate two imaging modalities. For example, systems have been developed for integrating CT and PET scanning capabilities, or CT and SPECT scanning capabilities, into partially or fully integrated combined imaging systems. In a partially integrated combined imaging system, the two imaging systems are mounted stationary in close proximity to one another (e.g., in a single room). A common subject table is mounted to a guide rail assembly in the floor and is constructed to be used with both imaging systems. The common subject table travels within the guide rail assembly to move the subject from the first imaging system to the second imaging system. In a fully integrated combined imaging system, the detectors of the two imaging modalities are mounted on a common gantry framework and are aligned with respect to a common Z-direction. A common subject table is moved along the common Z-direction through the detectors of one of the imaging systems to the detectors of the other imaging system.

Combined imaging systems cooperate to join at least two types of imaging data. Currently, in combined imaging systems, at least one of the scanning systems measures a distribution of an imaging agent, such as a radiopharmaceutical agent, that is introduced into the subject. The imaging agent is taken up by an organ until reaching a desired distribution after a limited time (e.g. several seconds to several minutes or hours) after introduction. In combined imaging systems, the subject may be off, or already be on, the common subject table while the imaging agent achieves the desired distribution. In accordance with certain conventional protocols, once the imaging agent achieves the desired distribution, both modalities in the fully integrated imaging system are then activated to simultaneously scan the region of interest. In this context, “simultaneous” scanning also includes successive scans separated by no more than a few minutes, such as when a subject is initially scanned in a PET or CT portion of the system followed within minutes by a scan in the other of the PET or CT portion of the system.

Partially and completely integrated systems afford certain advantages in connection with the collection of data related to different characteristics of a region of interest. When imaging a region of interest, certain aspects of the anatomical state within the region of interest may be of interest, as well as certain aspects of the functional state of the region of interest. Different imaging modalities exhibit different abilities to capture and present functional and anatomical state information. For example, X-ray, CT and MR systems acquire anatomical information with high spatial and temporal resolution. PET and SPECT scanners acquire functional information with generally high sensitivity and sometimes high specificity. Integrated dual-modality systems, whether partially or completely integrated, enable the collection of anatomical and functional state information for the region of interest at substantially a common point in time and thus the image data sets reflect functional and anatomical states for a common physiologic state.

However, partially and fully integrated imaging systems have certain limitations. For example, only one subject can be scanned at a time due to the reliance on a common subject table and the close proximity of the imaging systems to one another. Also, in a partially integrated system, a guide rail assembly must be installed to coordinate and control movement of the table between the two imaging systems. In a fully integrated system, a common detector framework must be constructed to support two types of detectors. Also, in order to implement a partially or completely integrated combined system, each imaging modality has an associated cost that is quite substantial. Thus, it is very costly to install a complete new system having two modalities that are partially or fully integrated with one another. Further, certain types of integrated dual-modality systems are difficult to design for operation so close to one another. For example, it is difficult to operate an ultrasound, X-ray, CT, PET or SPECT scanner in close proximity with an MR scanner given the magnetic fields introduced into the examination area surrounding the MR scanner.

A need remains for improved systems, protocols and methods for obtaining imaging data sets that are representative of concurrent functional and anatomical state of a region of interest.

### BRIEF DESCRIPTION OF THE INVENTION

In accordance with at least certain embodiments, methods, protocols and systems are provided for multi-modality imaging that comprising, among other things, introducing an imaging agent into a subject, the imaging agent configured to collect generally in a region of interest (ROI) in the subject during an uptake time interval and to maintain a pseudo-steady state (PSS) distribution within the ROI for a PSS time interval. The methods, protocols and systems also obtain a first image data set with a first imaging modality during a first acquisition time interval that occurs proximate in time with at least one of the uptake time interval or the PSS time interval. Alter the first acquisition time interval ends, the subject is transferred from the first imaging modality to a second imaging modality during a transfer time interval that overlaps the PSS time interval. Once transfer is complete, a second image data set is obtained with the second imaging modality during a second acquisition time interval that overlaps the PSS time interval in which the imaging agent maintains the PSS distribution in the ROI. The transfer time interval and second acquisition time interval substantially fall within the PSS time interval.

In accordance with at least one embodiment, the first acquisition time interval occurs coincident in time with the uptake time interval such that the first image data set reflects the physiologic state of the ROI during the uptake time interval. Optionally, the method, protocol and system may include altering a physiologic state of the ROI through at least one of exercise, injection of a pharmacological agent, and electrical stimulus. The second image data set is obtained while the imaging agent persists in the ROI and maintains the PSS distribution reflective of a first functional state of the ROI even after the ROI is no longer in the first functional state. The imaging agent represents at least one of a radiopharmaceutical (RP) agent or contrast agent used in imaging modalities other than nuclear medicine and PET.

Optionally, a protocol may be defined in connection with at least one of a brain perfusion study, a myocardial perfusion study, a whole body scan, a bone scan, a liver scan, a kidney scan, a lung scan, a brain scan, a cardiovascular scan, image guided therapy, assessment of myocardial tissue viability, ischemia analysis, a pulmonary study, tumor scans, infection scans, and a colonoscopy. As described hereafter, embodiments of the present invention are presented in connection with methods, protocols and systems for implementing multi-modality imaging based on predetermined pharmacokinetics of an imaging agent. The pharmacokinetics of the imaging agent is representative of the behavior of the imaging agent once externally administered to a living subject, such as a human or animal. The imaging agent's pharmacokinetics defines, among other things, a distribution of the imaging agent within one or more physiologic structures within the subject as a function of time.

In accordance with alternative embodiments, methods, protocols and systems are provided for imaging a region of interest (ROI) in a subject, the ROI uptaking an imaging agent while the ROI is in a physiologic state of interest at a first point in time to record a functional state of the ROI at the first point in time. The methods, protocols and systems comprise obtaining, during a first acquisition interval, a first image data set while the ROI is in the physiologic state of interest at the first point in time, the first image data set being obtained while the imaging agent has at least partially begun to collect in the ROI. During a second imaging interval, a second image data set is obtained based on the imaging agent in the ROI. The second image data set is obtained after the imaging agent has reached a pseudo-steady state (PSS) distribution in the ROT. The second image data set represents the functional state of the ROI at the first point in time.

### BRIEF DESCRIPTION OF THE DRAWINGS

FIG. 1 illustrates a multi-modality imaging system implemented in accordance with an embodiment of the present invention.

FIG. 2 illustrates a graph representative of the pharmacokinetics of exemplary imaging agents.

FIG. 3 illustrates a time line associated with an exemplary protocol that may be carried out in accordance with an imaging agent having known pharmacokinetics in accordance with an embodiment.

FIG. 4 illustrates a time line associated with another exemplary protocol that may be carried out in accordance with an imaging agent having known pharmacokinetics in accordance with another embodiment.

FIG. 5 illustrates a method or protocol for implementing a multi-modality imaging system in accordance with an embodiment of the present invention.

FIG. 6 illustrates a method or protocol for implementing a multi-modality imaging system in accordance with an alternative embodiment of the present invention.

FIG. 7 illustrates a multi-slice scanning imaging system, for example, a CT imaging system that may be utilized as the first or second imaging modality.

FIG. 8 is an isometric view of an embodiment of a PET imaging system that may be utilized as the first or second imaging modality.

FIG. 9 shows a block diagram of PET imaging system of FIG. 8.

### DETAILED DESCRIPTION OF THE INVENTION

Certain terms and phrases used throughout the present application shall be interpreted consistent with the explanations set forth herein.

The term “physiologic structure,” as used throughout, shall include any structure within a human or animal, such as bone, vasculature, nerves, an organ, a group of organs, or a portion of an organ, or a tumor, as well as any system or portion of a system within a human or animal. By way of example only, the physiologic structure may include all or a portion of the heart, brain, blood-brain barrier, lungs, liver, kidneys, lymph nodes, thyroid, stomach, thorax, neck, intestines, colon and the like. As a further example, the physiologic structure may include the pulmonary system, the nervous system, the vascular system, the blood pool, the renal system, the digestive system and any other system within an animal or human. The term “region of interest” or “ROI.” as used throughout, shall include all or any portion of one or more physiologic structures. By way of example, the ROI may represent the pulmonary system, the myocardium, a single chamber of the heart, the coronary artery, the colon, a tumor, an inflammation or any portion thereof and the like.

The term “physiologic state,” as used throughout, shall include both the functional state and the anatomical state of a given physiologic structure at a point in time. The term “physiologic state” includes neurostimulation states and normal and abnormal (pathophysiologic) states, including pathophysioligic states that are induced as part of a protocol or study (e.g., stress test, pharmacologic agent induced, electrically induced, etc.). By way of example, at least some physiologic structures within a subject undergo changes in physiologic state on regular or irregular bases. Changes in a physiologic state may occur periodically, intermittently or continuously. At any given point in time, a physiologic structure will exhibit a current instantaneous functional state and a corresponding current instantaneous anatomical state. The physiologic state of an ROI may be stable (e.g. constant for several minutes or hours) or transient (e.g., changing every few minutes or seconds). Physiologic states may change fast (e.g. within even heart beat or every breath) or slow (over the course of minutes, hours, days, weeks or months). The functional state of a physiologic structure includes, among other characteristics, the metabolic state and state of perfusion of the physiologic structure.

The term “imaging agent,” as used throughout, shall include any and all radiopharmaceutical (RP) agents and contrast agents used in connection with diagnostic imaging and/or therapeutic procedures. The imaging agent may represent a perfusion agent. The imaging agent may be, among other things, an imaging agent adapted for use in MRI or functional MRI, an intravenous CT contrast agent, a radiopharmaceutical PET or SPECT tracer, an ultrasound contrast agent, an optical contrast agent, myocardial perfusion tracers, cerebral perfusion tracer and the like. By way of example only, the imaging agent may be Myoview. Flourodeoxyglucose (FDG), ¹⁸F-Flourobenzyl Triphenyl Phosphonium (¹⁸F-FBnTP), ¹⁸F-Flouroacetate, ¹⁸F-labled myocardial perfusion tracers, Tc-ECD, Tc-HMPAO, N-13 ammonia, Envision N-13H3, Iodine-123 ligands, ^{99m}-Technitium ligands, Xenon-133, Neuroreceptor ligands, etc.). ¹⁸F-fluoromisonidazole, ²⁰¹Thallium, ^{99m}Technetium sestamibi, and ⁸²Rubidium and the like.

The term “imaging modality,” as used throughout, shall include any and all diagnostic imaging and therapeutic modalities including, but not limited to, positron emission tomography (PET), single photon emission composite tomography (SPECT), magnetic resonance imaging (MRI), functional MRI, computed tomography (CT), static X-ray imaging, dynamic X-ray imaging (fluoroscopy), ultrasound imaging, optical imaging, intravascular imaging, intravascular ultrasound imaging, intracardiac echo cardiography, electrophysiology, hemodynamics, MR guided focused ultrasound, CT guided focused ultrasound, myocardial ablation, radioactive seed implantation, ischemia detection and quantification and the like.

The term “pseudo-steady state distribution” or “PSS distribution,” as used throughout in connection with an imaging agent, shall include a range of distributions of the imaging agent where a sufficient amount of imaging agent remains in the region of interest to acquire an image data set based on the imaging agent. It is understood that different ranges of distributions will apply to different protocols and studies. For example, the range of distributions may vary based upon the imaging modality, imaging agent, region of interest, type of protocol and type of study. As one example, the distribution may be measured as the biological concentration of the imaging agent or metabolites of the imaging agent. A distribution will be considered pseudo-steady state, even though the distribution varies over time, so long as the distribution variation does not exceed an acceptable or predetermined limit. For example, in accordance with certain protocols, the distribution may be considered pseudo-steady state, even when the distribution varies or falls 25% below a maximum distribution level. As another example, in accordance with other protocols, the distribution may be considered pseudo-steady state, even when the distribution varies or falls 10% to 20% below a maximum distribution level. An acceptable range of variation, for the distribution to be considered pseudo-steady state, may depend upon a half-life of RP-type imaging agents, the sensitivity and/or specificity of the imaging modality, the protocol, the type of study and/or the region of interest.

Embodiments of the methods, systems and protocols described herein may be used in connection with a variety of different types of protocols and studies. For example, the study may be one or more of a brain perfusion study, a myocardial perfusion study, a whole body scan, a bone scan, a liver scan, a kidney scan, a lung scan, a brain scan, a cardiovascular scan, a pulmonary study, a tumor scan, an infection scan, a colonoscopy and the like. Also, the study may not simply be diagnostic in nature, but instead may be implemented in connection with treatment or therapy, such as implantation of radioactive seeds for cancer treatment, myocardial ablation, image guided therapy (e.g., ultrasound, MR or CT guided therapy utilizing focused ultrasound or radiation treatment) and the like. As another example, the study may be performed in connection with assessment of tissue viability following a stroke, heart-attack, vascular blockage, seizure, aneurysm and the like. The study may include operations to assess ischemic and infarcted tissue in the brain or myocardium.

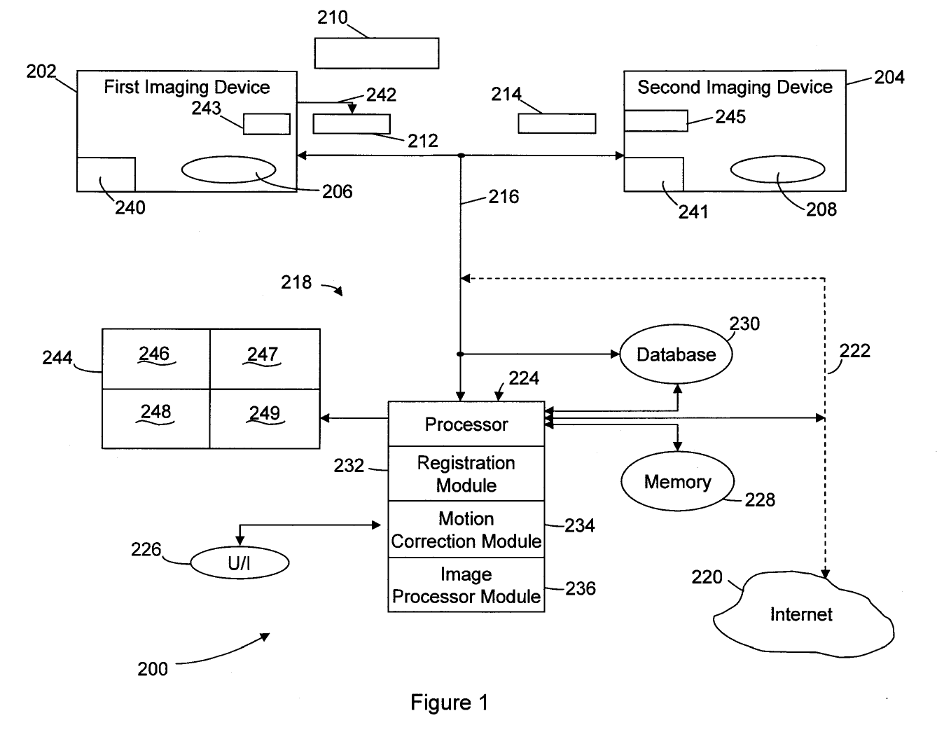

FIG. 1 illustrates a multi-modality imaging system implemented in accordance with an embodiment of the present invention. The system 200 includes first and second imaging systems or devices 202 and 204. The first imaging device 202 is configured to operation in a first imaging modality (e.g., X-ray, MR, CT, PET, SPECT, ultrasound, etc.) to obtain a first image data set 206 representative of the ROI during a first acquisition time interval. At least a portion of the first image data set is obtained while the ROI is in a first physiologic state and after an imaging agent reaches the PSS distribution in the ROI. The first data set records an anatomical state of the ROI at a given point in time while in a physiologic state of interest.

The imaging device 202 includes an injection mechanism 242 that is located proximate to a subject table 212. The injection mechanism 242 may be integral with, coupled to, or operate entirely separate from, the imaging device 202. The injection mechanism 242 controls the timing, amount and flow of imaging agent introduced into the subject. For example, the injection mechanism 242 may control an amount of a radiopharmaceutical (RP) agent, and/or contrast agent that in injected in a venous system of subject. The injection mechanism 242 may be manually controlled by an operator at the imaging device 202, or alternatively, may be automatically controlled in accordance with an imaging protocol 240 (e.g., pharmacokinetics driven injection of imaging agent). The injection mechanism 242 may also include the ability to inject a pharmacological agent (e.g. adenosine, diamox, insulin or other medication to change a myocardial perfusion state or a brain perfusion state). The imaging device 202 includes a motion detection device or monitor 243 (such as a pulmonary and/or ECG monitor) to optionally monitor characteristics representative of motion, such as breathing cycles of the subject and/or cardiac cycles, of the subject during the first acquisition time interval. Optionally, the monitor 243 may represent a transducer belt, or an ultrasound-based monitor that obtains ultrasound-based position or motion information regarding the subject or the region of interest. For example, ultrasound sensors may be located in the subject table, or configured to be mounted on, the patient during the first acquisition time interval by the first imaging 202. The ultrasound-based position information may be used to detect motion within the region of interest through segmentation and/or auto regression analysis and the like. Alternatively, the first imaging device 202 may represent an ultrasound diagnostic imaging scanner. When the first imaging device 202 is an ultrasound scanner, the diagnostic ultrasound image data set may be analyzed to identify and correct for motion in the region of interest. Optionally, the motion monitor 243 may be an MR-based detector, or an optical-based detector.

The second imaging device 204 is configured to operation in second imaging modality (e.g., PET, SPECT, ultrasound) to obtain a second image data set 208 during a second acquisition time interval. The second image data set is obtained while the imaging agent persists in the ROI and maintains the PSS distribution, even after the ROI is no longer in the physiologic state that existed during the first acquisition time interval. The first and second imaging devices 202 and 204 are located physically remote and separate from one another, such as in different rooms within a hospital or clinic, different buildings within a hospital, clinical or university campus and the like.

The subject being scanned is transferred from the first imaging device 202 to the second imaging device 204 between the scans. As explained below, a maximum preferred transfer time interval is established for the subject to be transferred from the first imaging device 202 to the second imaging device 204. The subject may be transferred on a table 210 that is movable separate and independent from either of the imaging devices 202 and 204. Optionally, the subject may be transferred with a wheelchair or simply by walking between the imaging devices 202 and 204. Each of the imaging devices 202 and 204 include respective subject imaging tables 212 and 214 that are coupled to the corresponding imaging device 202 and 204 and operated (e.g., moved in the X-direction, Y-direction, Z-direction, rotated, tilted and the like) in a coordinated manner with corresponding data acquisition operations. The second imaging device 204 includes a breath and/or ECG monitor 245 to monitor the patients breathing and/or cardiac cycle during the second acquisition time interval. The information collected by the monitors 243 and 245 may be used to correct for motion artifacts, and may be used to temporally and spatially register the first and second image data set with one another. The monitor 245 may be an ultrasound-based monitor as described above to obtain ultrasound-based position information, or an MR-based detector or an optical-based detector.

The imaging devices 202 and 204 are coupled over a network 216 to one another and to a workstation 218. Optionally, the imaging devices 202 and 204 may be coupled to the Internet 220 over a network link 222. The network 216 conveys, among other things, image data sets, protocols and the like between the imaging devices 202 and 204 and workstation 218. The workstation 218 includes one or more monitors 222 that are controlled by a processor module 224. The processor module 224 is coupled to a user interface 226 (e.g., keyboard, mouse, touch pad, etc.), a local memory 228 and an image database 230. The memory 228 and/or database 230 may be utilized to store one or more of the image data sets 206 and 208, as well as protocols, and the programming instructions to carry out the processes described herein. The processor module 224 performs various operations as described hereafter. For example, the processor module 224 may implement one or more of a registration module 232, a motion correction module 234, and an image processor module 236. The registration module 232 spatially and temporally registers the first and second image data sets 206 and 208 with one another. The motion correction module 234 detects and corrects artifacts in the image data sets 206 and 208 due to motion of the subject during data acquisition. The image processor module 236 processes the image data sets 206 and 208 to generate 3D images, 2D images, rendered images, fused images and the like. One or more monitors 244 are coupled to the processor module 224 and image processing module 236 to present the images in windows 246-249 on the monitor 244. While the workstation 218 is illustrated as a stand-alone device, the workstation 218 may be implemented with, or integrated into, either of the first and second imaging devices 202 and 204.

As described hereafter, embodiments of the present invention are presented in connection with methods, protocols and systems for implementing multi-modality imaging based on predetermined pharmacokinetics of an imaging agent. The pharmacokinetics of the imaging agent is representative of the behavior of the imaging agent once externally administered to a living subject, such as a human or animal. The imaging agent's pharmacokinetics defines, among other things, a distribution of the imaging agent within one or more physiologic structures within the subject as a function of time.

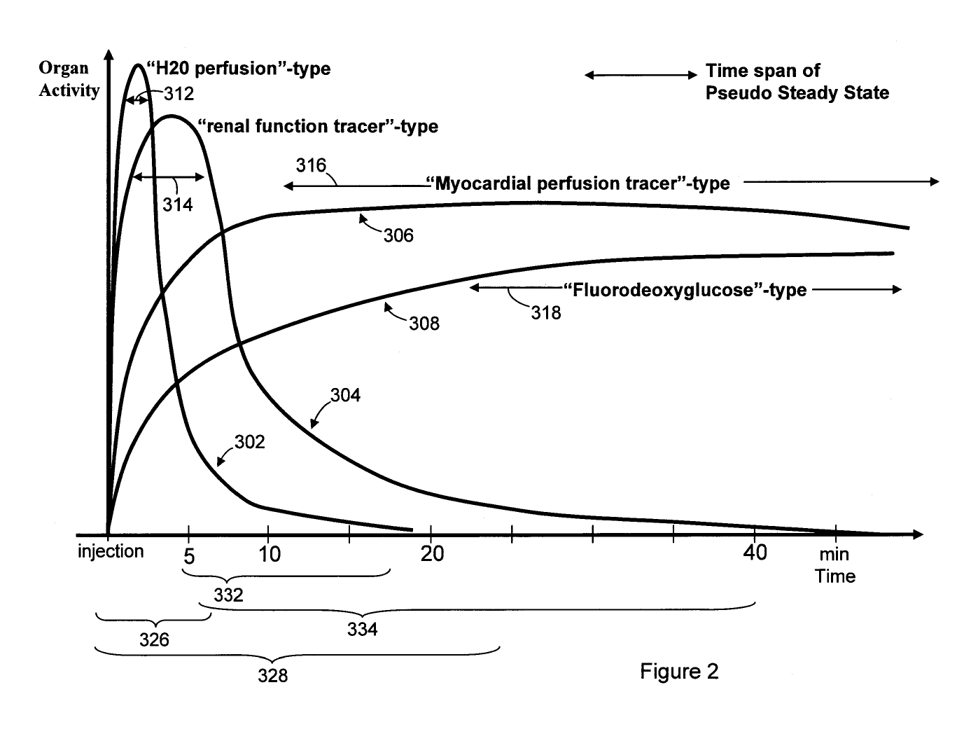

FIG. 2 illustrates a graph representative of the pharmacokinetics of exemplary imaging agents. The graph in FIG. 2 illustrates time along the horizontal axis and distribution of the imaging agent within a region of interest along the vertical axis. Imaging agents exist that, after introducing into the subject, reach a pseudo-steady state distribution within the region of interest within a few seconds, minutes or hours. FIG. 2 illustrates distributions of four different imaging agents. The imaging agent denoted by graph 302 corresponds to a water perfusion type of imaging agent. The graph denoted by reference numeral 304 corresponds to a renal function tracer, while the graphs denoted by reference numerals 306 and 308 correspond to imaging agents configured for use as a myocardial perfusion tracer and a FDG tracer, respectively.

As shown in FIG. 2, each distribution exhibits a sharp rise corresponding to an uptake interval or phase until tapering off and obtaining a maximum distribution value. After the distribution of the imaging agent attains the maximum, the high level distribution is maintained for a short or long period of time (depending upon the imaging agent) before falling off in connection with a wash-out interval or phase. The time interval in which the distribution of the imaging agent maintains a relatively high value within an acceptable range represents the pseudo-steady state time interval or phase. In the example of FIG. 2, arrows 312, 314, 316 and 318 denote the pseudo-steady state (PSS) time intervals or phases associated with the distributions of the imaging agents 302-308. The brackets 326 and 328 denote the uptake time intervals or phases associated with the imaging agents 306 and 308 preceding the PSS distributions 316 and 318, respectively. The brackets 332 and 334 denote the wash-out phases associated with the imaging agents 302 and 304.

As is apparent in FIG. 2, the water perfusion type imaging agent (graph 302) and renal function tracer (graph 304) exhibit PSS time intervals that are approximately 2 to 4 minutes and 6 to 8 minutes in length, respectively. Within five minutes of introduction, the water perfusion type imaging agent is substantially washed out. Within 10 minutes of introduction, the renal function tracer is substantially washed out. The myocardial perfusion tracer (graph 306) and the FDG tracer (graph 308) exhibit PSS time intervals that are over 30 minutes in length. The myocardial perfusion tracer and FDG tracer do not substantially wash out for well over 1 hour (not shown in FIG. 2).

In accordance with embodiments described herein, methods, protocols and systems are presented, by which two or more imaging systems or devices (e.g., MR-PET, CT-PET, Ultrasound-SPECT, etc.) acquire image data sets in a non-simultaneous manner, yet the image data sets record simultaneous states (e.g., anatomic state and functional state) that exist concurrently within a region of interest at a single point in time. Image acquisition times for the two or more imaging modalities are managed based on the pharmacokinetics of the imaging agent such that image data sets, which are acquired at different points in time, may be co-displayed to present simultaneous and concurrent anatomic and functional states.

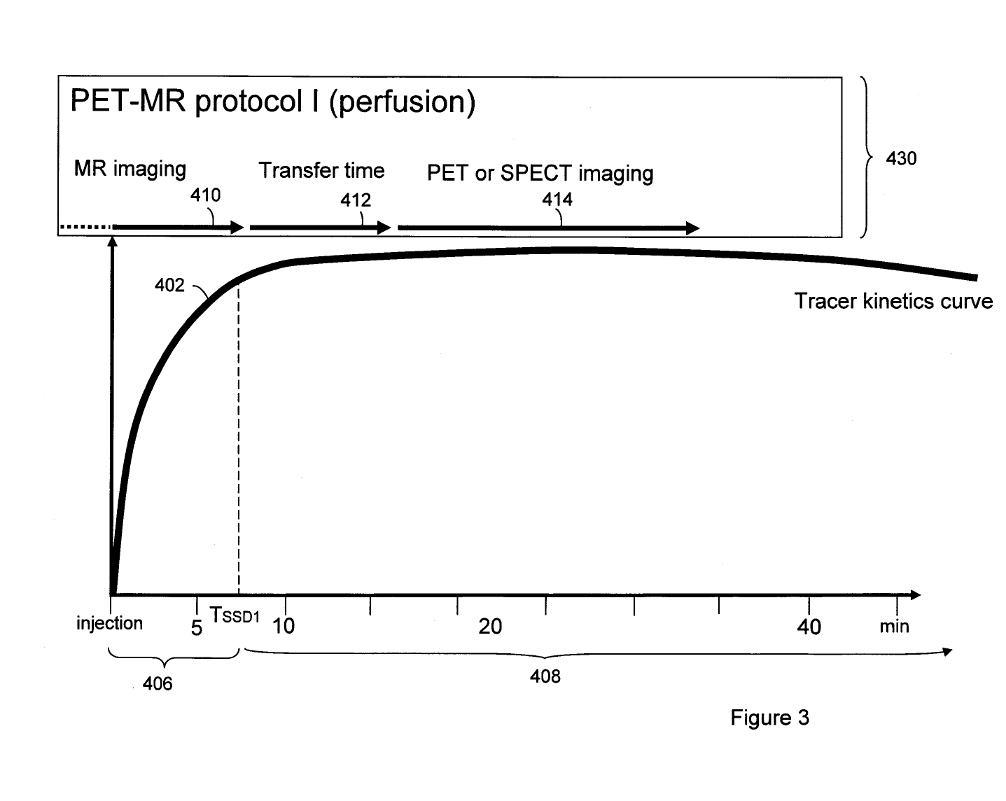

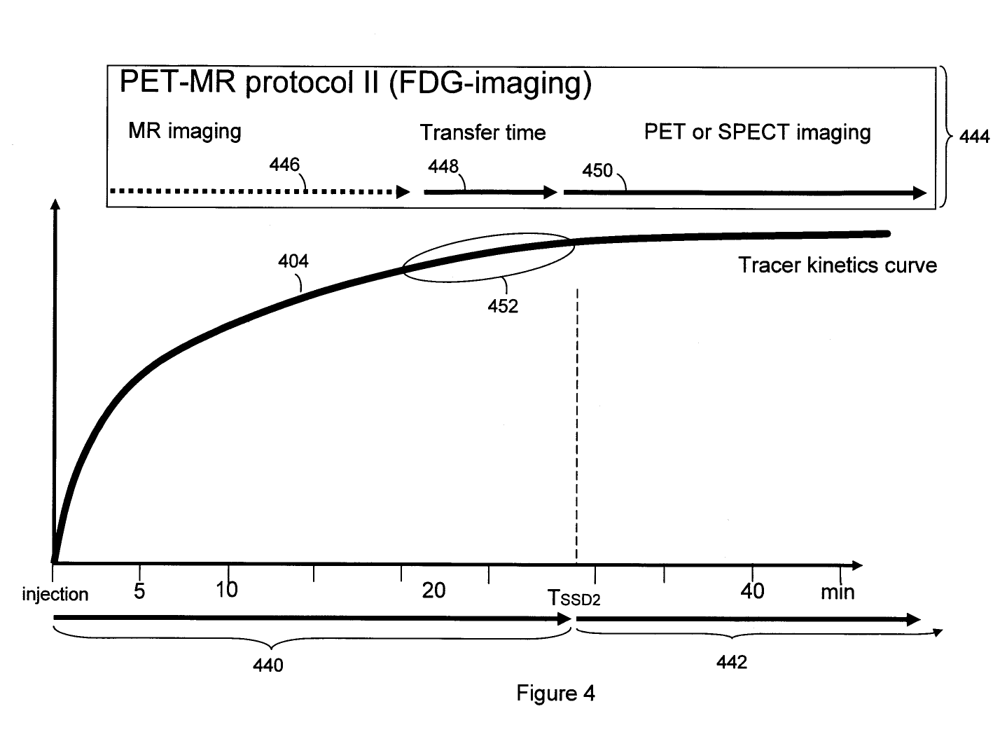

FIGS. 3 and 4 illustrate time lines associated with exemplary protocols that may be carried out in accordance with imaging agents having the illustrated pharmacokinetics. FIGS. 3 and 4 illustrate graphs having time along the horizontal axis and distribution (of the imaging agent within the region of interest) along the vertical axis. It should be recognized that the graphs 402 and 404 are for illustrative purposes only and are not representative of exact distributions of specific imaging agents. In FIG. 3, graph 402, illustrates the pharmacokinetics of an exemplary imaging agent that may be used in accordance with a PET/MR perfusion imaging study. In FIG. 4, graph 404, illustrates the pharmacokinetics for an exemplary imaging agent that may be used in accordance with a PET/MR FDG imaging study. The pharmacokinetics associated with perfusion imaging agent 402 (FIG. 3) are somewhat different from those of the FDG imaging agent 404 (FIG. 4). For example, the perfusion imaging agent 402 exhibits a much faster uptake behavior and achieves a pseudo-steady state distribution more quickly by the time denoted T_{SSD1}. The uptake time interval or phase associated with the perfusion imaging agent 402 is labeled 406, followed by a longer pseudo-steady state distribution time interval or phase labeled 408.

In the example of FIG. 3, the uptake time interval 406 may be between 5 and 10 minutes in length, and more specifically, approximately 8 minutes in length. The pseudo-steady state time interval 408 may extend for 50 to 75 minutes in length, or more specifically approximately 60 minutes in length. Optionally, perfusion imaging agents may be utilized having pseudo-steady state time intervals that extend well beyond 60 minutes in length, such as 2 hours in length or more. The perfusion imaging agent is collected in the ROI during the uptake time interval 406. The ROI uptakes the perfusion imaging agent while the ROI is in a first physiologic state to record a first functional state of the ROI at a particular point in time. The perfusion imaging agent 402 persists in the ROI and maintains the PSS distribution even after the ROI is no longer in the first functional state. For example, the myocardium will progress through numerous systolic and diastolic cycles over the course of a 30 minute to 2 hour PSS time interval. Thus, the myocardium will not remain in any functional state for a very long period of time. The imaging agent records or freezes the functional state of the myocardium at the point in time T_{SSD1} when attaining the PSS distribution. In at least one embodiment, the PSS distribution may represent an integration of the concentration of the imaging agent in the ROI over time. Therefore, once the PSS distribution is reached, small variations in the physiologic state, throughout the PSS time interval, will not substantively change the PSS distribution.

FIG. 3 also illustrates a time line associated with an exemplary protocol 430 for performing a PET/MR perfusion imaging study utilizing the perfusion imaging agent 402. According to the protocol 430, first, an MR imaging modality is implemented to obtain an MR image data set during an MR acquisition time interval 410. In the example of FIG. 3, the MR acquisition time interval 410 ends coincident with the end of the uptake time interval 406 and the beginning of the PSS time interval 408. The MR image data set is obtained while a region of interest (ROI) is in a first physiologic state. The MR imaging modality obtains at least a portion of the MR image data set while the perfusion imaging agent 402 has at least partially begun to collect in the ROI and thus record the functional state of the ROI. Upon completion of the MR acquisition time interval 410, the subject is transferred from the MR imaging modality to the PET imaging modality. A transfer time interval 410 is defined as a maximum preferred time interval for which the subject may take to transfer. It is recognized that the subject may take less time or slightly more time to transfer than the transfer time interval 412. By way of example, transfer time interval 412 may be 5 minutes or more, and more preferably 10 to 15 minutes, or some other sufficient time for the subject to get out of the MR imaging modality, switch rooms and lay down on the table (or sit on a chair) for the PET or SPECT imaging modality. Once the subject is transferred to the PET imaging modality, third, the PET image data set is obtained during the PET acquisition time interval 414 based on the imaging agent in the ROI.

It is recognized that the protocol 430 may include other operations before, during or after the operations discussed above. Optionally, the protocol 430 may include an operation to apply an external stress to the subject to alter the physiologic state of the region of interest to attain a desired physiologic state. For example, the external stress may be applied through exercise, by injection of a pharmaceutical agent, through electrical stimulus and the like.

Alternatively, the perfusion protocol 430 may be implemented by substituting SPECT imaging for the PET imaging. In this alternative example, the second imaging modality would represent a nuclear medicine acquisition system that obtains a SPECT image data set. The imaging agent and SPECT acquisition time interval may be modified accordingly.

The perfusion protocol 430 is defined based on the pharmacokinetics of the imaging agent represented by graph 402. It is recognized that other protocols may be defined based on the pharmacokinetics of the imaging agent represented by graph 402. The perfusion protocol 430 initiates the MR acquisition time interval 410 sufficiently early to obtain a desired MR image data set before, during, or shortly after, time T_{SSD1}. The MR acquisition time interval 410 may begin before, concurrent with, or after the point in time at which the perfusion imaging agent 402 is injected. The MR acquisition time interval 410 occurs proximate in time with the pseudo-steady state time interval 408, such as by completing the MR acquisition time interval 410 substantially at the same time as, shortly before or shortly after, the perfusion imaging agent 402 reaches the pseudo-steady state distribution at time T_{SSD1}. By way of example only, if the perfusion imaging agent 402 is expected to achieve a pseudo-steady state distribution approximately 8 minutes after the injection point in time, the MR acquisition time interval 410 may be defined to terminate within 5-15 minutes after the imaging agent is injected, more specifically within 7-10 minutes after the imaging agent is injected, and even more specifically at approximately 8 minutes after the imaging agent is injected. In the example of FIG. 3, the transfer time interval 412 is defined to be approximately 10 minutes in length. Optionally, alternative transfer time intervals may be established, such as when the pseudo-steady state time interval 408 is longer or shorter, as well as when the PET acquisition time interval 414 is longer or shorter. In the example of FIG. 3, the PET acquisition time interval 414 may be between 10 and 50 minutes in length. In the example of FIG. 3, the PET acquisition time interval 414 is approximately 20 minutes in length. Alternatively, the PET acquisition time interval 414 may be much shorter (e.g. 1-5 minutes, or 5-10 minutes) or longer (e.g. 40-75 minutes).

FIG. 4 illustrates a time line associated with another exemplary protocol that may be carried out in accordance with the imaging agent 404. In FIG. 4, the illustrative graph 404 is representative of an imaging agent which exhibits a longer uptake time interval 440 (than the uptake time interval 406 in FIG. 3) before reaching a pseudo-steady state distribution at time T_{SSD2}. Upon achieving the pseudo-steady state distribution at time T_{SSD2}, the FDG imaging agent 404 maintains the pseudo-steady state for a PSS time interval 442 that is longer (e.g. 1-2 hours) than the PSS time interval 408 in FIG. 3. In the example of FIG. 4, the FDG imaging agent 404 has an uptake time interval 440 of approximately 30 minutes followed by a PSS time interval 442 of one to two hours.

Based on the pharmacokinetics of the FDG imaging agent 404, an FDG protocol 444 is defined in which an MR acquisition time interval 446 is established, followed by the transfer time interval 448, followed by a PET or SPECT acquisition time interval 450. The MR acquisition time interval 446, in accordance with the FDG protocol 444, may be initiated after the injection point in time, at which the FDG imaging agent 404 is injected, given the relatively long uptake time interval 440. The MR acquisition time interval 446 terminates at a point in time that occurs proximate in time with the beginning of the PSS time interval 442. In particular, the MR acquisition time interval 446 is coincident with the uptake time interval 440. In the example of FIG. 4, the MR acquisition time interval 446 terminates approximately 7-10 minutes (which is still considered proximate in time) before the PSS time interval 442 begins at time T_{SSD2}. The transfer time interval 448 is defined in the FDG protocol 444 to also fall prior to the time at which the PSS time interval 442 begins. The MR acquisition time interval 446 and transfer time interval 448 may be both established prior to the PSS time interval 442 due to the relatively flat or slowly increasing uptake characteristic, at section 452 in the graph 404. The flat or slowly increasing uptake behavior at section 452 of the graph 404 indicates that a substantial majority of the FDG imaging agent has already collected within the region of interest during the MR acquisition time interval 446 prior the transfer time interval 448. Given that the substantial majority of the FDG imaging agent accumulated within the region of interest during the MR acquisition time interval 446, the FDG imaging agent will record or freeze the functional state of the region of interest substantially near, or at the conclusion of the MR acquisition time interval 446. While the FDG imaging agent continues to be uptaken through the subject transfer time 448, the additional amount of FDG imaging agent uptake during the section 452 of the graph 402 will not substantively impact the functional state recorded by the FDG imaging agent. Thus, the MR acquisition time interval 446 is considered to occur proximate in time with the beginning of the PSS time interval 442.

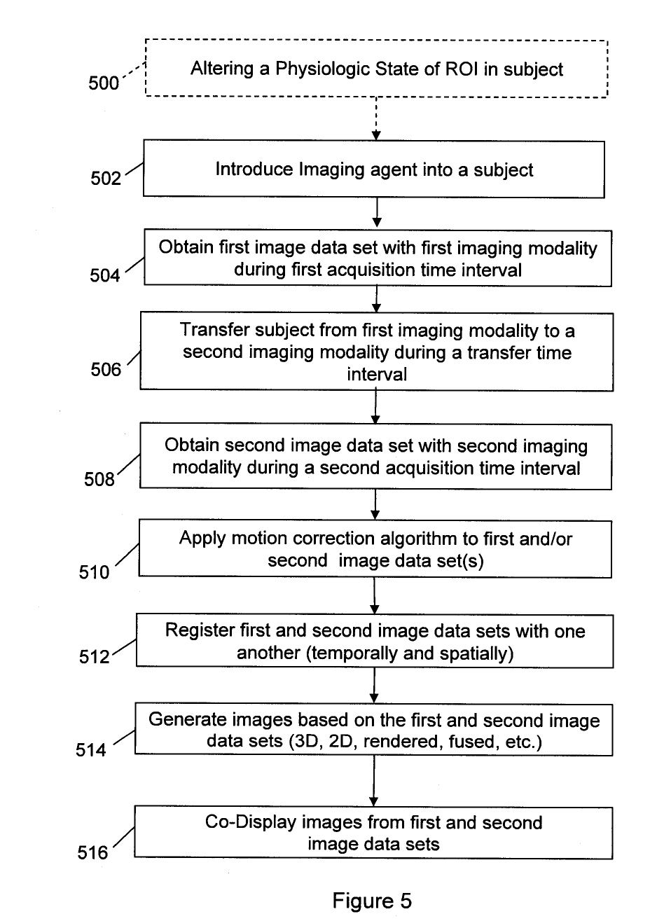

FIG. 5 illustrates a method or protocol for implementing a multi-modality imaging system in accordance with an embodiment of the present invention. At 500 a physiologic state of the ROI is altered such as through at least one of exercise, injection of a pharmacological agent, and electrical stimulus. Before or after 500, the subject is placed on the table of the first imaging modality. At 502, an injection mechanism is used to introduce an imaging agent into a subject. The imaging agent may represent a radiopharmaceutical (RP) agent, a contrast agent, a perfusion agent and the like. The imaging agent is designed to collect in a region of interest in the subject and to maintain a pseudo-steady state (PSS) distribution in the ROI for a pseudo steady state time interval.

Once the imaging agent is introduced, flow moves to 504 at which a first imaging modality obtains a first image data set during a first acquisition time interval. The first acquisition time interval occurs proximate in time with at least a beginning of the PSS time interval. For example, the first acquisition time interval may partially overlap the PSS time interval. The imaging agent is collected in the ROI during an initial uptake time interval that precedes the PSS time interval. In at least one embodiment, the first imaging modality may obtain all or a portion of the first image data set during the uptake time interval. As a further example, in studies having very short uptake time intervals, the first acquisition time interval may substantially precede introduction of the image agent into the subject. As a further option, all or a substantial portion of the first image data set may be obtained after the imaging agent reaches the pseudo-steady state distribution. The first imaging modality acquires the first image data set utilizing a scalable property that is separate and distinct from any emission properties of the imaging agent. The first image data set is representative of at least certain characteristics of the physiologic state (e.g. An anatomical and/or functional state) of the ROI, where such characteristics are measurable through the use of scannable property that is utilized by the first imaging modality.

For example, in CT and X-ray scans, the CT or X-ray image data set is acquired utilizing attenuation measurements of transmissions through the subject. A CT or X-ray image data set that is obtained from the ROI is representative of the anatomical state of the ROI at the time that the CT or X-ray image data set is obtained. The acquisition time interval over which the CT or X-ray image data set is obtained may be very short (e.g., a few seconds) or relatively long (e.g., a several minutes).

Returning to FIG. 5, once the first image data set is obtained, the subject is transferred from the first imaging modality to the second imaging modality. The time period needed to transfer the subject from the first imaging modality to the second imaging modality is defined as the maximum preferred transfer time interval. The beginning time and end time of the transfer time interval are defined relative to the injection point in time. Optionally, during 506, the actual transfer time interval may be tracked (e.g., with a timer) and/or the total time from introduction of the imaging agent. The actual transfer time interval may then be recorded with the image data sets and/or used to modify the second acquisition time interval. The transfer time interval overlaps, in time, with the PSS time interval in which the pseudo-steady state distribution of the imaging agent is maintained within the region of interest. The subject may be transferred immediately upon completion of the first acquisition time interval. The subject may be transferred by moving a table from one room within the facility to another room between the first and second imaging modalities. The table holding the subject may be configured for use in one or both of the first and second imaging modalities. Alternatively, separate and independent tables may be used with each of the first and second imaging modalities, in which case the subject would be transferred to a transition gurney and/or required to walk between the rooms containing the first and second imaging modalities.

Once the subject is placed in the second imaging modality, at 508, a second image data set is obtained by scanning the subject during a second acquisition time interval. By way of example, the second imaging modality may represent a PET scanner, a SPECT scanner or a nuclear medicine scanner configured to perform both PET and SPECT scans. Optionally, the second imaging modality may be any other type of scanner that is configured to detect properties associated with imaging agent. The second image data set is obtained during the second acquisition time interval that is temporally aligned to overlap the PSS time interval in which the imaging agent maintains the pseudo-steady state distribution in the region of interest. The second image data set is obtained while the imaging agent maintains the pseudo-steady distribution, which may be after the ROI is no longer in the physiologic state of interest (e.g., functional state, metabolic state or perfused state). A beginning time and end time of the second acquisition time interval are defined relative to the injection point in time.

Optionally, the beginning time of the second acquisition time interval may be modified based on the end time of the transfer time interval. For example, when an actual transfer time interval is longer or shorter than a predetermined transfer time interval, the beginning time of the second acquisition time interval may be similarly moved forward or backward in time. Optionally, the end time of the second acquisition time interval may be modified based on the beginning time of the second acquisition time interval and/or the end time of the transfer time interval. For example, when i) an actual transfer time interval is longer or shorter than a predetermined transfer time interval or ii) the beginning time of the second acquisition time interval is moved forward or backward in time, the end time of the second acquisition time interval may be similarly moved forward or backward in time. The amount that the beginning and end times of the second acquisition time interval may be moved is based on the pharmacokinetics of the imaging agent.

Once the imaging agent is introduced into the ROI, the imaging agent acts to temporarily record (e.g. over 10 minutes, 20-60 minutes, 50 minutes, 2 hours, etc.) the functional state or condition of the ROI while the ROI was in the physiologic state of interest. The imaging agent reaches the pseudo-steady state distribution within the ROI and persists within the ROI for a period of time. The concentration of the imaging agent will slowly diminish. As the concentration of the imaging agent slowly diminishes, the imaging agent maintains the pseudo-steady state distribution that is representative of a particular functional state of the ROI when in the physiologic state of interest. The pseudo-steady state distribution of the imaging agent persists and remains even after the ROI changes physiologic state.

At 508, the second imaging modality obtains the second image data set at a time that is independent of, and without regard for, the current physiologic state of the ROI. By maintaining the pseudo-steady state distribution of the imaging agent for a period of time following uptake, the second imaging modality is afforded an opportunity to measure, later in time, an earlier functional state of the ROI.

At 510, the first and second image data sets may be analyzed to determine whether motion occurred during acquisition in one or both image data sets. When an undesirable amount of motion occurs during data acquisition, it may be desirable to apply a motion correction algorithm. At 510, the motion correction algorithm may be applied to one or both of the first and second image data sets. Motion correction may be based on ECG or breath information collected by the monitors 243 and 245 (FIG. 1). Optionally, the motion correction may be based on ultrasound information, MR information, optical information and the like. For example, when the first image modality represents an ultrasound or MR scanner, the ultrasound or MR scanner may obtain position information in addition to, or as part of, the ultrasound or MR diagnostic imaging data set. For example, the position information may be derived from segmentation and/or autocorrelation analysis of the ultrasound diagnostic image data set. As a further option, ultrasound, magnetic or optical sensors may produce motion information separate from the first imaging modality. Optionally, the first image data set may be corrected for motion immediately after being obtained at 504. Alternatively, the first image data set, if scanned in a sufficiently short period of time, may be less susceptible or not susceptible to motion. Thus, the first image data set may not warrant correction for motion.

At 512, the first and second image data sets are registered spatially and temporally with one another. Spatial registration may be achieved through manual means, such as through an operator who selects matching points within images from each of the first and second image data sets. Alternatively, the first and second image data sets may be spatially registered with one another automatically through software based on landmark detection, segmentation, auto-regression analysis and the like. Temporal registration between the first and second data sets may be based on ECG or breath information collected by the monitors 243 and 245.

At 514, images are generated based upon the first and second image data sets. Various types of images may be generated. For example, three-dimensional images may be presented for co-display based on the first and second image data sets. In addition or alternatively, the images may include two-dimensional images, individual orthogonal slices, cut planes, surface rendered images, volume rendered images and/or fused images. The images may be grey scale or color, or a combination thereof. The images may be organized into cine-loops to present motion of the ROI over time. The images may be rotated, tilted, or otherwise adjusted while displayed. Images from the first and second image data sets are produced and co-displayed in a manner such that functional states measured in the second image data set are co-displayed with concurrent anatomical metabolic or functional states measured in the first image data set, where the functional, anatomical and metabolic states all occurred simultaneously within the region of interest. At 516, the images are then co-displayed.

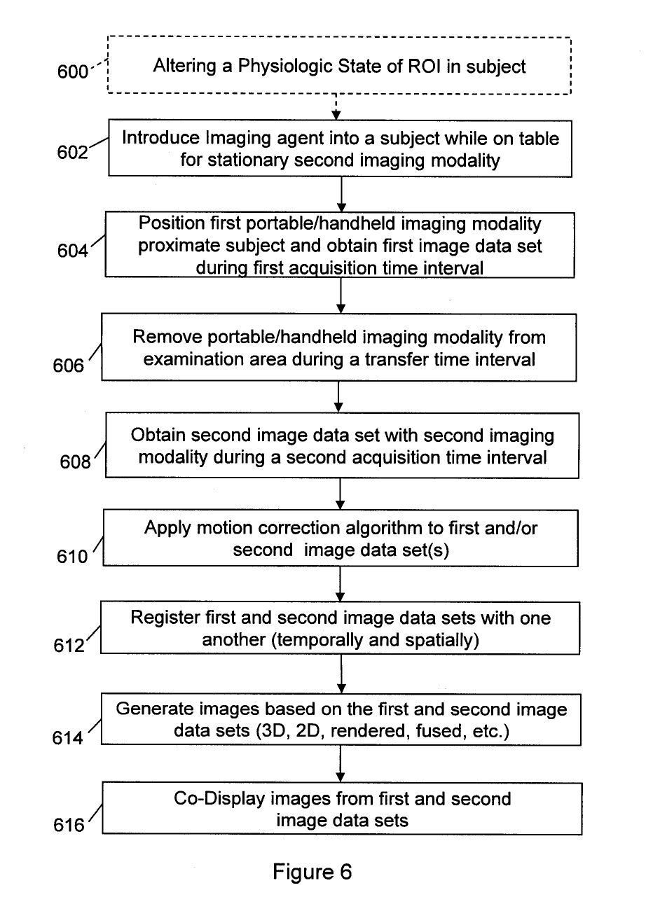

FIG. 6 illustrates a method for implementing a multi-modality imaging system in accordance with an alternative embodiment of the present invention. In the embodiment of FIG. 6, one or both of the first and second imaging modalities may represent portable or handheld scanners. For example, a portable scanner may represent an ultrasound, PET, CT, X-ray, optical or nuclear medicine scanner on a wheeled cart. As another option, the portable scanner may constitute a portable C-arm type X-ray or CT scanner on wheels. Alternatively, a handheld scanner may represent a laptop computer coupled to an ultrasound probe, a catheter, an optical probe, an X-ray probe, a handheld PET scanner, a handheld nuclear medicine scanner and the like.

When a portable or handheld scanner is used as one or both imaging modalities, the subject may remain in a single room, common bed, or same general examination area throughout both image acquisition time intervals. Instead, the transfer may occur by the portable or handheld scanner being moved in and out of the room or examination area where the subject is located. In one exemplary system, the first imaging modality may constitute a portable or handheld scanner and the second imaging modality may constitute a stationary PET or nuclear medicine scanner.

In the example of FIG. 6, at 600 a physiologic state of the ROI is altered such as through at least one of exercise, injection of a pharmacological agent, and electrical stimulus. Before or after 600, the subject is placed on the table of the stationary second imaging modality. At 602, an injection mechanism is used to introduce an imaging agent into a subject. Before or while the imaging agent is being uptaken into the ROI, at 604, the portable or handheld first imaging modality is positioned proximate to the subject in a desired position and orientation relative to the ROI. The first image data set is obtained during a first acquisition time interval. At 606, the portable/handheld imaging modality is removed from the subject and examination area during a transfer time interval. In the example of FIG. 6, the subject does not move during the transfer time interval. Instead, the portable/handheld imaging modality is transferred away from the subject.

At 608, the second image data set is obtained using the second imaging modality (e.g., a stationary PET or nuclear medicine scanner). At 610, motion correction is applied to the first and/or second image data sets. At 612, the first and second image data sets are registered (spatially and/or temporally) with one another. At 614, images are generated based on the first and second image data sets and at 616 the generated images are displayed.

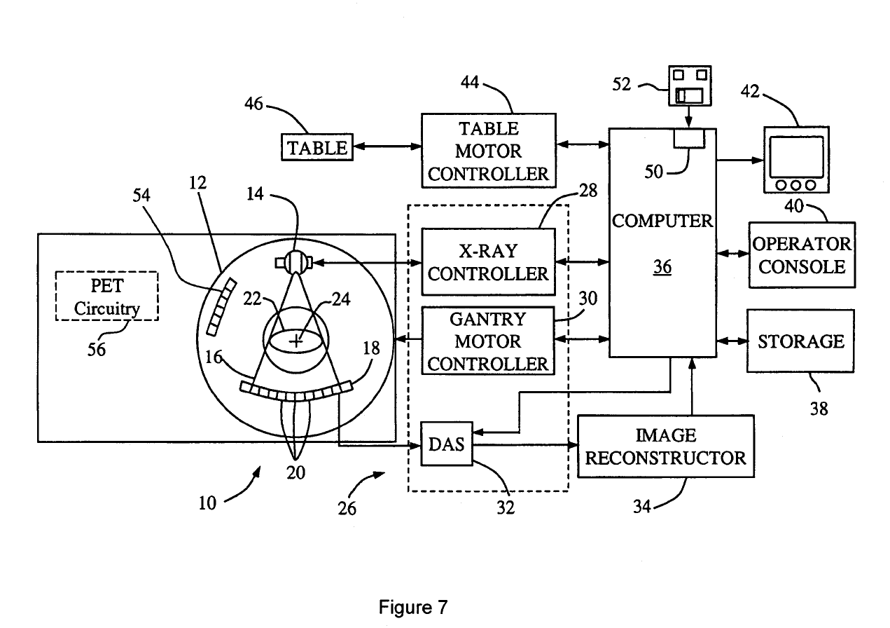

FIG. 7 illustrates a multi-slice scanning imaging system, for example a CT imaging system that may be utilized as the first or second imaging modality. Gantry 12 has an X-ray source 14 that projects a beam of X-rays 16 toward a detector array 18 on the opposite side of gantry 12. Detector array 18 is formed by a plurality of detector rows (not shown) including a plurality of detector elements 20 which together sense the projected X-rays that pass through an object, such as a medical subject 22. Each detector element 20 produces an electrical signal that represents the intensity of an impinging X-ray beam and hence allows estimation of the attenuation of the beam as it passes through object or subject 22. During a scan to acquire X-ray projection data, gantry 12 and the components mounted thereon rotate about a center of rotation 24.

FIG. 7 shows only a detector row of detector elements 20. However, multi-slice detector array 18 includes a plurality of parallel detector rows of detector elements 20 such that projection data corresponding to a plurality of quasi-parallel or parallel slices can be acquired simultaneously during a scan.

Rotation of gantry 12 and the operation of X-ray source 14 are governed by a control mechanism 26 of CT system 10. Control mechanism 26 includes an X-ray controller 28 that provides power and timing signals to X-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized X-ray data from DAS 32 and performs high-speed image reconstruction. The reconstructed image is applied as an input to a computer 36 which stores the image in a storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, X-ray controller 28 and gantry motor controller 30. In addition, computer 36 operates a table motor controller 44 which controls a motorized table 46 to position subject 22 in gantry 12. Particularly, table 46 moves portions of subject 22 through gantry opening 48. The computer 36 may implement the operations discussed above in connection with FIGS. 2-6. The computer 36 may include the workstation 218 of FIG. 1.

In one embodiment, computer 36 includes a device 50, for example, a floppy disk drive or CD-ROM drive, for reading instructions and/or data from a computer-readable medium 52, such as a floppy disk or CD-ROM. In another embodiment, computer 36 executes instructions stored in firmware (not shown). Computer 36 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein.

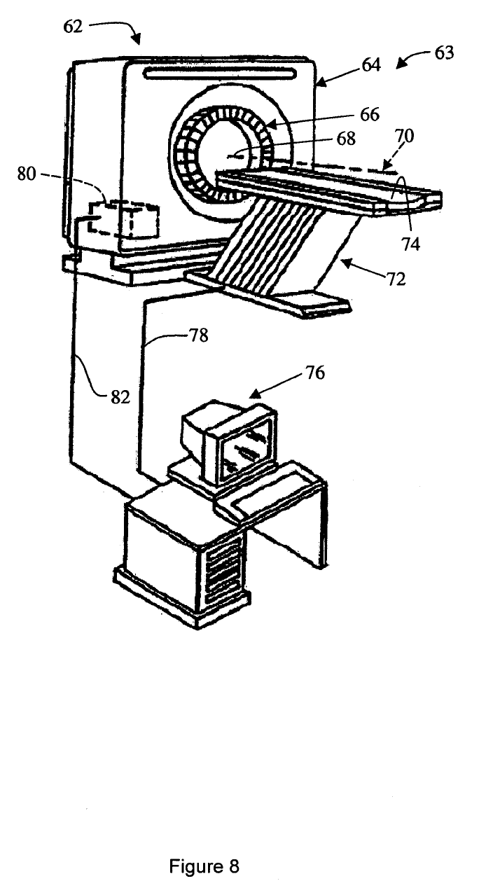

FIG. 8 is an isometric view of an embodiment of a PET imaging system that may be utilized as the first or second imaging modality. PET imaging system 62 includes a PET scanner 63. PET scanner 63 includes a gantry 64 which supports a detector ring assembly 66 about a central opening, or bore 68. Detector ring assembly 66 is circular in shape, and is made up of multiple detector rings (not shown) that are spaced along a central axis 70 to form a cylindrical detector ring assembly. A table 72 is positioned in front of gantry 66 and is aligned with central axis 70 of detector ring assembly. A table controller (not shown) moves a table bed 74 into bore 68 in response to commands received from an operator work station 76 through a serial communications link 78. A gantry controller 80 is mounted within gantry 64 and is responsive to commands received from operator work station 76 through a second serial communication link 82 to operate gantry 64.

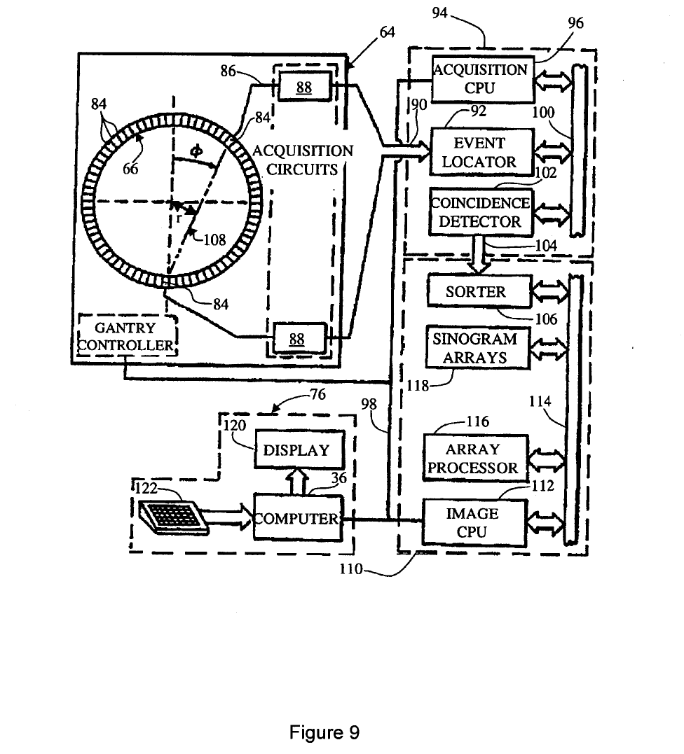

FIG. 9 shows a block diagram of PET imaging system of FIG. 8. Each detector ring of detector ring assembly 66 includes detectors 84. Each detector 84 includes scintillator crystals (not shown). Each scintillator crystal is disposed in front of a photomultiplier tube (PMT) (not shown). PMTs produce analog signals on line 86 when a scintillation event occurs at one of the scintillator crystals that are disposed in front of the PMTs. The scintillation event occurs when a photon is received by one of the scintillator crystals. In one embodiment, photons are generated by administering a compound, such as, ¹¹C-labeled glucose, ¹⁸ F-labeled glucose, ¹³N-labeled ammonia and ¹⁵O-labeled water within the object, an emission of positrons by the compounds, a collision of the positrons with free electrons of the object, and generation of simultaneous pairs of photons. Alternatively, the photons are transmitted by rotating rod sources within a FOV of PET imaging system 62. A set of acquisition circuits 88 is mounted within gantry 64 to receive the signals and produce digital signals indicating event coordinates (x,y) and total energy. These are sent through a cable 90 to an event locator circuit 92 housed in a separate cabinet. Each acquisition circuit 88 also produces an event detection pulse (EDP) which indicates the exact moment the scintillation event took place.

Event locator circuits 92 form part of a data acquisition processor 94 which periodically samples the signals produced by acquisition circuits 88. Processor 94 has an acquisition central processing unit (CPU) 96 which controls communications on a local area network 98 and a backplane bus 100. Event locator circuits 92 assemble the information regarding each valid event into a set of digital numbers that indicate precisely when the event took place and the position of a scintillation crystal which delected the event. This event data packet is conveyed to a coincidence detector 102 which is also part of data acquisition processor 94. Coincidence detector 102 accepts the event data packets from event locators 92 and determines if any two of them are in coincidence. Events which cannot be paired are discarded, but coincident event pairs are located and recorded as a coincidence data packet that is conveyed through a serial link 104 to a sorter 106.

Each pair of event data packets that is identified by coincidence detector 102 is described in a projection plane format using our variables r, v, θ, and Φ. Variables r and Φ identify a plane 108 that is parallel to central axis 70, with Φ specifying the angular direction of the plane with respect to a reference plane and r specifying the distance of the central axis from the plane as measured perpendicular to the plane. Variables v and θ (not shown) further identify a particular line within plane 108, with θ specifying the angular direction of the line within the plane, relative to a reference line within the plane, and v specifying the distance of center from the line as measured perpendicular to the line.

Sorter 106 forms part of an image reconstruction processor 110. Sorter 106 counts all events occurring along each projection ray, and stores that information in the projection plane format. Image reconstruction processor 110 also includes an image CPU 112 that controls a backplane bus 114 and links it to local area network 98. An array processor 116 also connects to backplane bus 114. Array processor 116 converts the event information stored by sorter 106 into a two dimensional sinogram array 118. Array processor 116 converts data, such as, for instance, emission data that is obtained by emission of positrons by the compound or transmission data that is obtained by transmission of photons by the rotating rod sources, from the projection plane format into the two-dimensional (2D) sinogram formal. Examples of the 2D sinogram include a PET emission sinogram that is produced from emission data and a PET transmission sinogram that is produced from transmission data. Upon conversion of the data into the two-dimensional sinogram format, images can be constructed. Operator work station 76 includes computer 36, a cathode ray tube (CRT) display 120, and a keyboard 122. Computer 36 connects to local area network 98 and scans keyboard 122 for input information. Through keyboard 122 and associated control panel switches, the operator controls calibration of PET imaging system 62, its configuration, and positioning of table 72 for a PET scan. Similarly, once computer 36 receives a PET image and a CT image, the operator controls display of the images on CRT display 120. On receipt of the PET image and the CT image, computer 36 performs a method for combining an anatomic structure and metabolic activity for an object, such as subject 22. The computer 36 of the PET imaging system 62 may implement the operations discussed above in connection with FIGS. 2-6. The PET imaging system 62 may include or be coupled to the workstation 218 of FIG. 1.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third.” etc, are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. A method for multi-modality imaging, comprising:
introducing an imaging agent into a subject, the imaging agent configured to collect generally in a region of interest (ROI) in the subject during an uptake time interval and to maintain a pseudo-steady state (PSS) distribution within the ROI for a PSS time interval;
obtaining a first image data set with a first imaging modality during a first acquisition time interval that occurs proximate in time with at least one of the uptake time interval and the PSS time interval;
transferring the subject from the first imaging modality to a second imaging modality during a transfer time interval that overlaps the PSS time interval; and
obtaining a second image data set with the second imaging modality during a second acquisition time interval that overlaps the PSS time interval in which the imaging agent maintains the PSS distribution in the ROI.

2. The method of claim 1, wherein the first acquisition time interval is coincident with the uptake time interval such that the first image data set reflects a physiologic state of the ROI during the uptake time interval.

3. The method of claim 1, further comprising altering a physiologic state of the ROI through at least one of exercise, injection of a pharmacological agent, and electrical stimulus.

4. The method of claim 1, wherein the transfer time interval and second acquisition time interval substantially fall within the PSS time interval.

5. The method of claim 1, wherein at least a portion of the first image data set is obtained while the ROI is in a first physiologic state and after the imaging agent reaches the PSS distribution, the second image data set being obtained while the imaging agent persists in the ROI and maintains the PSS distribution even after the ROI is no longer in the first physiologic state.

6. The method of claim 1, wherein the imaging agent represents at least one of a radiopharmaceutical (RP) agent and contrast agent used in imaging modalities other than nuclear medicine and PET.

7. The method of claim 1, wherein the first and second imaging modalities acquire the first and second image data sets utilizing anatomical and functional scannable properties, respectively.

8. The method of claim 1, wherein the imaging agent or metabolites of the imaging agent attains an upper concentration and thereafter maintains a biological concentration within the ROI that does not fall below the upper concentration by more than 20% throughout the PSS time interval.

9. The method of claim 1, wherein the injecting, transferring and obtaining operations are performed based on a protocol defined in connection with at least one of a brain perfusion study, a myocardial perfusion study, a whole body scan, a bone scan, a liver scan, a kidney scan, a lung scan, a brain scan, a cardiovascular scan, image guided therapy, assessment of myocardial tissue viability, ischemia analysis, a pulmonary study, a tumor scan, an infection scan, and a colonoscopy.

10. A method for imaging a region of interest (ROI) in a subject, the ROI uptaking an imaging agent while the ROI is in a physiologic state of interest at a first point in time to record a functional state of the ROI at the first point in time, the method comprising:
obtaining, during a first acquisition interval, a first image data set while the ROI is in the physiologic state of interest at the first point in time, the first image data set being obtained while the imaging agent has at least partially begun to collect in the ROI; and
obtaining, during a second imaging interval, a second image data set based on the imaging agent in the ROI, the second image data set being obtained after the imaging agent has reached a pseudo-steady state (PSS) distribution in the ROI, the second image data set representing the functional state of the ROI at the first point in time.

11. The method of claim 10, wherein the first image data set records one of an anatomical state and a functional state of the ROI at the first point in time.

12. The method of claim 10, wherein the first acquisition interval coincides in time with the ROI being in the physiologic state of interest, while the second imaging interval occurs later in time when the ROI is no longer in the physiologic state of interest.

13. The method of claim 10, wherein the first image data set is based on a scannable property that is unrelated to a distribution of the imaging agent in the ROI.

14. The method of claim 10, wherein the second image data set is obtained at a point in time after the first point in time without regard for a current physiologic state of the ROI.

15. The method of claim 10, wherein a current functional state of the ROI during at least a portion of the second imaging interval differs from the functional state recorded by the imaging agent in the PSS distribution.

16. A multi-modality imaging system, comprising:
an injection mechanism for introducing an imaging agent into a subject, the imaging agent configured to collect generally in a region of interest (ROI) in the subject during an uptake time interval and to maintain a pseudo-steady state (PSS) distribution in the ROI for a PSS time interval;
a first imaging modality configured to obtain a first image data set during a first acquisition time interval that occurs proximate in time with at least one of the uptake time interval and the PSS time interval;
a second imaging modality configured to obtain a second image data set during a second acquisition time interval that overlaps the PSS time interval in which the imaging agent maintains the PSS distribution in the ROI, the first and second imaging modalities being located physically separate from one another such that the subject must be transferred from the first imaging modality to the second imaging modality during a transfer time interval that overlaps the PSS time interval.

17. The system of claim 16, wherein the first acquisition time interval is coincident with the uptake time interval such that the first image data set reflects a physiologic state of the ROI the uptake time interval.

18. The system of claim 16, wherein the injection mechanism introduces the imaging agent at a desired time relative to the ROI attaining a physiologic state of interest, such that the PSS distribution records a functional state of the ROI when in the physiologic state of interest.

19. The system of claim 16, wherein the first imaging device obtains at least a portion of the first image data set after the imaging agent reaches the PSS distribution while the ROI is in a first functional state and wherein the second imaging modality obtains the second image data set representative of the first functional state after the ROI is no longer in the first functional state.

20. The system of claim 16, further comprising a display to co-display images based on the first and second image data sets as at least one of a fused image, 2D images, 3D images, and rendered images.

21. The system of claim 16, wherein the first imaging modality is one of an MR, CT, ultrasound, optical scanner and X-ray scanner and the second imaging modality is one of a PET and SPECT scanner.

22. A system for imaging a region of interest (ROI) in a subject, the ROI uptaking an imaging agent while the ROI is in a first state to record a first functional state of the ROI, the system comprising:
a first imaging modality configured to obtain, during a first acquisition interval, a first image data set while the ROI is in the first physiologic state, the first imaging modality obtaining the first image data set while the imaging agent has at least partially begun to collect in the ROI; and
a second imaging modality configured to obtain, during a second imaging interval, a second image data set based on the imaging agent in the ROI, the second imaging modality obtaining the second image data set after the imaging agent has reached a pseudo-steady state (PSS) distribution in the ROI, the second image data set recording the functional state of the ROI while in the first physiologic state.

23. The system of claim 22, wherein the first acquisition device records the first image data set as one of an anatomical state and a functional state of the ROI while in the physiologic state of interest.

24. The system of claim 22, wherein the first acquisition interval coincides in time with the ROI being in the physiologic state of interest, and the imaging acquisition interval occurring later in time when the ROI is no longer in the physiologic state of interest.

25. The system of claim 22, wherein the first acquisition device obtains the first image data set based on a scannable property that is unrelated to a distribution of the imaging agent in the ROI.

26. The system of claim 22, wherein the imaging acquisition device obtains the second image data set at a point in time independent of a current physiologic state of the ROI.
